(19) United States
(12) Patent Application Publication
(10) Pub. No.: US 2023/0221297 A1
(43) Pub. Date: Jul. 13, 2023

(54) METHODOLOGY OF ACCURACY AND PRECISION DETERMINATION FOR A NEW URINE SEDIMENT ANALYZER

(71) Applicant: Siemens Healthcare Diagnostics Inc., Tarrytown, NY (US)

(72) Inventor: Kausik Das, Stoughton, MA (US)

(73) Assignee: Siemens Healthcare Diagnostics Inc., Tarrytown, NY (US)

(21) Appl. No.: 18/001,725

(22) PCT Filed: Jun. 16, 2021
(86) PCT No.: PCT/US2021/037603

§ 371 Date: Dec. 14, 2022

Related U.S. Application Data
(60) Provisional application No. 63/040,837, filed on Jun. 18, 2020.

(51) Int. Cl. G01N 33/493; G01N 35/00; G01N 15/14

(52) U.S. Cl. CPC G01N 33/493; G01N 35/00; G01N 15/14; G01N 2015/0065

(57) ABSTRACT

Non-limiting embodiments of a methodology of validating accuracy and precision determinations of a new urine sediment analyzer, and method(s) related thereto.

### CROSS REFERENCE TO RELATED APPLICATIONS

The subject application claims benefit under 35 USC § 119(e) of U.S. Provisional Application No. 63/040,837, filed Jun. 18, 2020. The entire contents of the above-referenced patent application(s) are hereby expressly incorporated herein by reference.

### STATEMENT REGARDING FEDERALLY FUNDED RESEARCH OR DEVELOPMENT

Not Applicable

### TECHNICAL FIELD

The presently disclosed and claimed inventive concept(s) relate to a method(s) for validating the accuracy and precision determinations of a new urine sediment analyzer.

### BACKGROUND

Urinalysis is essential to the diagnosis and treatment of patients suspected of various renal and urinary tract disorders. Urine samples of such suspected patients contain various analytes that are indicative of a pathological condition or stage of disease progression. For example, urine samples from patients with kidney stones or urinary tract injury contain red blood cells; urine samples from patients with various inflammatory conditions or urinary tract infections contain white blood cells and/or bacteria. Accurate and timely results of urinalysis is critical for patient care.

Urinalysis is the physical, chemical, and microscopic examination of urine. Urinalysis involves a number of tests to detect and measure various analytes that pass through urine. Physical examination of urine samples provides information regarding its appearance, such as color, odor, and turbidity. Chemical examination of urine provides information regarding acidity (pH), specific gravity, and presence of proteins, sugars, ketones, bilirubin, nitrite, white blood cell products (leukocyte esterase), and blood. Microscopic examination of urine provides information regarding sediments present in urine, such as, for example, red blood cells, white blood cells, bacteria, epithelial cells, pathological casts, hyaline cast, crystals, yeast, mucus, and sperm.

Microscopic examination includes microscopy-based sediment urinalysis, which is a common detection technology utilized by clinical laboratories. Microscopy-based urine sediment analyzers evaluate urine samples for the presence of analytes based on an analyte's unique morphological characteristics including size, shape, and textural appearance. Microscopy-based detection technologies depend entirely on the morphological aspects of the formed elements present in a urine sample—no chemical reactions are involved in microscopy-based detection of urine sediment particles. Urine sediment elements may be detected manually using microscopy, or detected automatically using various automated sediment urine analyzers that utilize microscopy-based image technology.

Standard automated urine sediment analyzers are based on either (1) image-based, or (2) flow cytometry technological and analytical principles. Image-based analysis systems depend solely on the microscopic morphological aspects of the sediment elements present in a urine sample and require no chemical reactions to detect analytes. There are two basic designs for image-based systems: flow cell and cuvette (sample holder) systems. Flow cell image-based automated urine sediment analyzers analyze urine sediment in dynamic fluid (rather than a static surface as in the cuvette-based method, as discussed below). A urine sample is forced to flow through a flow cell and digital images are captured of the flowing urine sample when stroboscopic light flashes. An algorithm is then used to count and classify sediment particles based on morphological characteristics. In cuvette image-based automated urine sediment analyzers, a camera captures microscopic images of a urine sample in a cuvette and similarly relies on an algorithm to count and classify sediment particles based on morphological characteristics. Unlike image-based systems, flow cytometry technologies depend on photochemical interactions and light scatter properties. More specifically, the photochemical interactions are between added fluorescent dye and the biochemical contents in a urine sample. As the sample pass through a flow cytometer, particles in the sample are illuminated by a laser and the elements in the flow cell are classified according to electrical impedance, light scatter, and fluorescence.

However, there are often discrepancies when establishing accuracy characteristic determinations between analyzers utilizing image-based technology and those utilizing flow cytometry, particularly when validating a new analyzer by comparing it to an older analyzer using a different technology. There are also discrepancies when establishing precision characteristic determinations for an analyzer being tested against itself. Accuracy characteristic determination is a measure of the degree of closeness of a measured or calculated value to its actual value. Precision characteristic determination involves measuring the same sample over multiple times to determine the repeatability and reproducibility of the results. Repeatability is defined by multiple measurements performed under repeatable conditions such as same analyzer, same operators, same operating conditions (temperature, pressure) and same laboratory. Reproducibility is defined by multiple measurements performed under reproducible conditions such as different analyzers, different operators, different locations, and conditions. In both analyzer types (i.e., analyzers utilizing image-based technology and those utilizing flow cytometry), the first step in validating a new analyzer is to preserve and/or store a urine sample that will be analyzed. However, preservation, storage, and preparation conditions affect analyzer results.

More specifically, discrepancies and poor correlation of urine sediment analysis results between the two different technologies occur due to several reasons. One such reason is each technology's reliance on differing characteristics of urine sediment. Urine sediment in a urine sample may deteriorate due to aging and undergo morphological changes as a result, which may affect the urine sediment analysis results, depending on the type of technology employed by the urine sediment analyzer. For example, white blood cells deteriorate in an aging urine sample. Image-based analyzers are reported to detect fewer white blood cells in an aging urine sample because aging white blood cells may have a different morphology compared to typical white blood cells. However, white blood cells in an aging urine sample may be correctly detected by flow cytometry-based detection technology due to the fact that the nucleic acid content of the white blood cells remain intact resulting in similar fluorescence even after deterioration of white blood cells due to aging. Even amongst image-based technologies, such as flow-cell analyzers and cuvette analyzers, morphological changes contribute to discordant results. (Wah D T, Wises P K, Butch A W. Analytical performance of the iQ200 automated urine microscopy analyzer and comparison with manual counts using Fuchs-Rosenthal Cell Chambers. Clin Chem 2005; 123: 290-296). For example, red blood cells may hemolyze or crenate in an aging urine sample. A cuvette-image based analyzer may correctly detect aging red blood cells, but a flow-cell based analyzer may not detect aging red blood cells because flow-cell based cameras may not be able to capture the unique morphology of aging red blood cells. Similarly, flow cytometry-based analyzers may also fail to detect aging red blood cells because such analyzers may register a different light scatter for a hemolyzed red blood cell (compared to a typical red blood cell), which would cause the flow cytometry-based analyzer to miss detecting red blood cells.

Another reason that causes discrepancies between analyzers utilizing image-based technology and those utilizing flow cytometry is the use of various pre-analytical sample preparation steps. For example, inadequate sample mixing prior to analysis is reported to cause disagreement between flow cell image-based systems and manual microscopic based methods. Inadequate mixing may, for example, leave white blood cell clumps intact in samples analyzed by a manual microscopy method, whereas mixing techniques applied by flow cell image-based analyzers may break the clump into individual cells resulting in the flow cell image-based analyzer to report a higher white blood cell count than that reported by the manual method. Additionally, the centrifugation step to concentrate urine samples prior to analysis for a manual microscopic counting method is attributed to discordant results between manual microscopic counting method and flow cytometry methods. (Ko D H, Jl M, Kim S, Cho E J, Lee W, Yun Y M, Chun S, Min W K. An approach to standardization of urine sediment analysis via suggestion of a common manual protocol. Scand J Clin Lab Invest 2016; 76: 256-263). Additionally, the stabilizing conditions can interact with the analytical steps of various analyzers differently. For example, a chemical that can stabilize a urine sample may interfere with the analytical steps involving chemical reactions in a flow cytometry-based analyzer but may not affect the analytical steps of an image-based analyzer. Alternatively, low temperature conditions used to stabilize cellular deterioration can result in a turbid sample that interferes with the image acquisition of an image-based analyzer but may not affect the chemical reaction of the flow cytometry based method.

The above reasons for such discrepancies among urine sediment analyzers are rooted in three factors: (a) difference in technological basis of urine sediment analyzers; (b) the heterogenous nature of urine sediment composition; and (b) the heterogenous stability profile of each sediment analyzer which impacts the measurements differently for different technologies.

Regarding factor (a) and as stated above, flow cell and cuvette technologies are image-based and depend on morphological aspects of sediment analytes wherein no chemical interaction or light scatter is involved. On the other hand, flow cytometry technologies are based on photochemical interactions and light scatter properties. Therefore, in flow cytometry systems, an imbalance of certain chemical properties of a urine sample—e.g., pH, certain medications, or urine preservatives—can inhibit dye binding chemical reactions, while the same imbalance may not have any effect on image-based technologies. For example, presence of ethanol in the urine matrix is reported to cause false detection of red blood cells, epithelial cells, cast, and bacteria in flow cytometry systems. It is also reported that the presence of undissolved particles can cause false positive detection of red blood cells by flow cytometry systems. (Delanghe J., Speeckaert M. Preanalytical requirements of urinalysis. Biochemia Medica 2014; 24: 89-104).

Regarding factor (b), the heterogenous nature of urine composition can also affect analyzers utilizing image-based technology and those utilizing flow cytometry differently. For example, sediment elements with similar morphological characteristics may cause interference in image-based methods but not in flow cytometry methods. For specific example, the presence of yeast is reported to cause false elevated levels of red blood cells in image-based analyzers. (Aydin O, Ellidag H Y, Eren E, Yilmaz N. High false positive and false negative yeast parameter in an automated urine sediment analyzer. J Med Biochem 2015; 34: 332-337).

Regarding factor (c), the different stability profiles of urine sediment elements can also affect analyzers utilizing image-based technology and those utilizing flow cytometry differently. Cellular elements such as red blood cells, white blood cells, and epithelial cells lyse at different rates and degrees. The rate of lysis is highest with red blood cells and lowest with epithelial cells. The rate of lysis is slower at refrigerated temperatures (2-8° C.). Microorganisms such as bacteria and yeast grow with time. The rate of growth is dependent on the storage temperature and urine composition. Crystals are reported to grow at higher rates at a refrigerated temperature due to decreased solubility at lower temperatures. However, urine sediment results from image-based analyzers have been found to be in agreement with reference groups when samples were stored without preservatives up to 8 hours at room temperature. (Ercan M, Akbulut E D, Abusouglu S, Yilmaz F M, Oguz E F, Topcuglu C, Oztekin V, Bogdaycioglu N. Stability of urine specimens stored with and without preservatives at room temperature and on ice prior to urinalysis. Clin Biochem 2015; 48: 919-922). In another study, strip tests for red blood cells and white blood cells yielded false negative results when the samples were stored at room temperature for 24 hours. (Froom P, Bieganiec B, Ehrenrich Z, Barak M. Stability of common analytes in urine refrigerated for 24 hours before automated analysis by test strips. Clin Chem 2000; 46: 1384-1386). It has been also been found that sediment results from both flow cytometry and image-based analyzers are stable for up to 2 hours at room temperature. (Manoni F, Valverde S, Caleffi A, Alessio M G, Silvestri M G, Rosa R D, Zugno A, Ercolin M, Gessoni G. Stability of common analytes and urine particles stored at room temperature before automated analysis. Int J Lab Med 2008; 4: 192-198).

Current accuracy characteristic determinations of new urine sediment analyzers by comparing various performance characteristics of the new analyzer to an older predecessor analyzer that uses the same technology (i.e., image-based technology or flow cytometry technology) can mislead the true performance determination. This strategy often has been used in manufacturing validations for various analyzers. Similar strategy is also used by hospital laboratories when upgrading to a newer version of an older analyzer. The new analyzer often provides improved overall performance, but the core technology remains the same. However, because the new analyzer and predecessor share the same technological principles, both the new analyzer and the comparative analyzer have similar outcomes—including similar errors—resulting in seemingly high correlation agreement. Such high agreement masks drawbacks of the new analyzer. Therefore, validation of a new analyzer against an established or reference analyzer that uses different technology may better reveal true performance capability. However, this approach requires that the interfering conditions be eliminated so that a true performance comparison can be evaluated. Therefore, there is a need for sample preservation, storage, and preparation conditions that allow new analyzers to be validated against any type of established or reference analyzer.

Precision characteristic determinations are also established for new analyzer during performance validation by the manufactures and by hospital laboratories. Both repeatability and reproducibility measurements require that the sample composition and sample integrity remain constant and stable during the multiple measurement procedures. This sample stability requirement often poses challenges because biological specimens often undergo various biochemical changes. Therefore, the samples used to measure repeatability and reproducibility precision are stabilized—i.e., preserved, stored, and prepared—by various methods. There is a need for sample preservation, storage, and preparation conditions that prevent interference by stabilizing conditions or stabilizing chemicals during the analytical steps of the measurement.

Therefore, when making both accuracy and precision characteristic determinations, there is a need for sample preservation, storage, and preparation conditions that provide accurate and precise determinations when validating new analyzers.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting. In the following detailed description of embodiments of the inventive concept, numerous specific details are set forth in order to provide a more thorough understanding of the inventive concept. It will be apparent to one of ordinary skill in the art, however, that the inventive concept within the disclosure may be practiced without these specific details. In other instances, well-known features have not been described in detail to avoid unnecessarily complicating the instant disclosure.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the systems, and/or methods disclosed and claimed herein can be made and executed without undue experimentation in light of the present disclosure. While the methods of this presently disclosed and claimed inventive concept(s) have been described in terms of preferred embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The singular forms “a,” “an,” and “the” include plural referents unless the context clearly indicates otherwise. Thus, for example, reference to “a compound” may refer to 1 or more, 2 or more, 3 or more, 4 or more or greater numbers of compounds. The term “plurality” refers to “two or more.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example, but not by way of limitation, when the term “about” is utilized, the designated value may vary by ±20% or ±10%, or ±5%, or ±1%, or ±0.1% from the specified value, as such variations are appropriate to perform the disclosed methods and as understood by persons having ordinary skill in the art. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

Further, use of the term “plurality” is meant to convey “more than one” unless expressly stated to the contrary.

As used herein any reference to “one embodiment” or “an embodiment” means that a particular element, feature, structure, or characteristic described in connection with the embodiment is included in at least one embodiment. The appearance of the phrase “in one embodiment” in various places in the specification are not necessarily all referring to the same embodiment. Further, all references to one or more embodiments or examples are to be construed as non-limiting to the claims.

The term “patient” includes human and veterinary subjects. In certain embodiments, a patient is a mammal. In certain other embodiments, the patient is a human. “Mammal” for purposes of treatment refers to any animal classified as a mammal, including human, domestic and farm animals, nonhuman primates, and zoo, sports, or pet animals, such as dogs, horses, cats, cows, etc.

As used herein, the term “processor” may refer to one or more units for processing including (for example but not by way of limitation) an ASIC, microcontroller, FPGA, microprocessor, digital signal processor (DSP) capability, state machine, or other suitable component. A processor may be configured to execute a computer program, e.g. as contained in machine readable instructions stored on a non-transitory computer readable memory and/or programmable logic. The processor may have various arrangements corresponding to those discussed for the circuitry, e.g. on-board and/or off board the apparatus as part of the system.

Turning now to particular embodiments, the presently disclosed and/or claimed inventive concept(s) relate generally to composition(s) and method(s) for preservation, storage, and preparation of urine samples for validating performance characteristics such as accuracy and precision determination of new urine sediment analyzers. More specifically, the presently disclosed composition(s) and method(s) relate to detecting disparities between the detection results of two different urine sediment analyzers, and particularly the results of two different urine sediment analyzers utilizing different technology principles. The present teachings are directed to composition(s) and method(s) capable of testing multiple elements, analytes, and characteristics of a urine sample. In addition, the present teachings provide for a composition and/or method that produces consistent data for various types of urine sediment analyzers, as opposed to requiring a distinct composition and/or method for each analyzer.

The method for validating the accuracy characteristic determinations of an automated urine sediment analyzer may comprise:
analyzing one or more urine samples for one or more analytes of interest, using a first automated sediment analyzer within a time period of less than or equal to 10 hours from collection of the one or more urine samples, wherein the one or more urine samples are selected from:
   (a) urine sample (A) is not subject to a temperature of below 8° C. prior to analyzing urine sample (A);
   (b) urine sample (B) has not had a preservative chemical applied to urine sample (B) prior to analyzing urine sample (B);
   (c) urine sample (C) was treated using predetermined separation techniques prior to analyzing urine sample (C);
   (d) urine sample (D) has had a preservative chemical applied to urine sample (D), and further wherein urine sample (D) was treated using predetermined storage conditions prior to analyzing urine sample (D); or
   (e) any combination of (a)-(d);
obtaining detection results of the one or more analytes of interest for the one or more urine samples from the first automated sediment analyzer;
analyzing the same one or more urine samples as analyzed by the first automated sediment analyzer for the one or more analytes of interest using a second automated sediment analyzer;
obtaining detection results of the one or more analytes of interest for one or more urine samples from the second automated sediment analyzer;
comparing the detection results of the one or more analytes of interest for the one or more urine samples from the first automated sediment analyzer and the second automated sediment analyzer to determine any deviation between the two detection results;
validating the accuracy characteristic determinations of the first automated sediment analyzer by determining whether the deviation between the detection results of the first sediment analyzer and the second automated sediment analyzer exceeds a predefined threshold level.

The method for validating the precision characteristic determinations of an automated urine sediment analyzer may comprise:
analyzing one or more urine samples for one or more analytes of interest, using a first automated sediment analyzer two or more times within a time period of less than or equal to 10 hours from collection of the one or more urine samples, wherein the one or more urine samples are selected from:
   (i) urine sample (1) has had a preservative chemical applied to urine sample (1);
   (ii) urine sample (2) was treated using predetermined storage conditions prior to analyzing urine sample (2);
   (iii) any combination of (i)-(ii);
obtaining detection results of the one or more analytes of interest for one or more urine samples from the first automated sediment analyzer for the two or more times that the first automated sediment analyzer was used to analyze the one or more urine samples;
comparing the detection results of the one or more analytes of interest for the one or more urine samples from each of the two or more times that the first automated sediment analyzer was used to determine any deviation between the two or more times that the first automated sediment analyzer was used;
validating the precision characteristic determinations of the first automated sediment analyzer by determining whether the deviation between the two or more times that the first automated sediment analyzer was used exceeds a predefined threshold level.

The inventive concepts disclosed herein may employ any method or system as the urine sediment analyzer to identify, classify, and count the formed elements in a urine sample using morphological methods. A variety of methods and analyzer systems for detecting analytes are well known in the art and include, but are not limited to, manual microscopy methods and automated analyzers. In certain non-limiting embodiments, the analyzer system is an automated urinalysis analyzer, which analyzer may utilize image-based technology or flow cytometry. Non-limiting examples of automated urine sediment analyzers that can be utilized in accordance with the present disclosure include Atellica® 1500 Automated Urinalysis System from Siemens Healthcare GmbH (Germany), UriSed2 from 77 Elektronika (Hungary), UriSed3 Pro from 77 Elektronika (Hungary), iQ200 Series Urinalysis Microscopy Analyzers from Beckman Coulter, Inc. (United States of America), sediMAX conTRUST Pro from A. Menarini Diagnostics (Italy), cobas® 6500 urine analyzer series from Roche Diagnostics (Switzerland), and cobas® u 701 microscopy analyzer from Roche Diagnostics (Switzerland).

The method(s) disclosed and/or claimed herein may be used for the validation of any automated urine sediment analyzer. The first urine sediment analyzer (i.e., the analyzer being validated for its accuracy and precision) may be selected from an image-based urine sediment analyzer and a flow cytometry-based image analyzer. The second urine sediment analyzer (i.e., the established or reference analyzer) may be selected from an image-based urine sediment analyzer, a flow cytometry-based image analyzer, and a manual microscopy analyzer. In some non-limiting embodiments, the second automated sediment analyzer utilizes a detection technology different than that of the first sediment analyzer.

Urine sediment analyzers, including manual and automated urine sediment analyzers, identify, classify, and count a variety of analytes including, but not limited to, red blood cells, white blood cells (including white blood cell clumps), bacteria (including, but not limited to, rod and coccus bacteria), squamous epithelial cells, non-squamous epithelial cells, pathological casts, hyaline casts, crystals, yeast, mucus, and sperm. Automated analyzers may also detect other characteristics of urine samples including, but not limited to, clarity, color, pH, specific gravity, bilirubin, glucose, ketone, leukocyte esterase, nitrite, protein, urobilinogen, albumin, creatine, albumin-to-creatine ratio, and protein-to-creatine ratio.

The presently disclosed and/or claimed inventive concept(s) teaches validating accuracy characteristic determinations by running one or more urine samples (urine samples (A)-(E)), or combinations thereof, over first and second urine sediment analyzers and comparing the obtained detection results from the one or more urine samples (urine samples (A)-(E)), or combinations thereof, in order to validate the first urine sediment analyzer. The presently disclosed and/or claimed inventive concept(s) also teaches validating precision characteristic determinations by running one or more urine samples (urine samples (1)-(2)), or combinations thereof, over a first urine sediment analyzer two or more times and comparing the obtained detection results from the one or more urine samples (urine samples (1)-(2)), or combinations thereof, from the selected times in order to validate the first urine sediment analyzer.

Each of urine samples (A)-(D), or combinations thereof, and each of urine samples (1)-(2) is analyzed within a time period of less than or equal to 10 hours from collection of the respective urine sample. It is desirable to maintain the time period from collection to analysis in order to maintain typical analyte morphology for purposes of detection. In certain non-limiting embodiments, each of urine samples (A)-(D), or combinations thereof, and each of urine samples (1)-(2) is analyzed within a time period of less than or equal to 8 hours from collection of the respective urine sample, or less than or equal to 4 hours from collection of the respective urine sample, or less than or equal to 2 hours from collection of the respective urine sample.

Urine sample (A) is analyzed by the first and second automated sediment analyzers and is not subject to a temperature of below 8° C. prior to analyzing urine sample (A). In certain non-limiting embodiments, urine sample (A) is not subject to a temperature of below 10° C. prior to analyzing urine sample (A), or less than 8° C. prior to analyzing urine sample (A), or less than 4° C. prior to analyzing urine sample (A), or less than 2° C. prior to analyzing urine sample (A)

Urine sample (B) has not had a preservative chemical applied to urine sample (B) prior to analyzing urine sample (B).

Urine sample (C) was treated using predetermined separation techniques prior to analyzing urine sample (C). Separation techniques may include centrifugation and filtration. In certain non-limiting embodiments, the second sediment analyzer utilizes an image-based detection technology or a flow cytometry-based detection technology, and further wherein urine sample (C) is subjected to a separation technique prior to analysis of urine sample (C). In other non-limiting embodiments, the second sediment analyzer utilizes a manual microscopy detection technology, and further wherein urine sample (C) is not subjected to a separation technique prior to analysis of urine sample. In certain non-limiting embodiments, the separation technique is centrifugation.

Urine sample (D) and urine sample (1) have had a preservative chemical applied to urine sample (D) and urine sample (1). In certain embodiments, urine sample (D) and urine sample (2) were treated using predetermined storage conditions prior to analyzing urine sample (D) or urine sample (2).

The preservative chemical depends on the type of urine sediment analyzer being used, whether as the new urine analyzer being validated, or as the reference analyzer. In certain non-limiting embodiments, the first sediment analyzer utilizes an image-based detection technology, and further wherein the preservative chemical is selected from non-interfering preservative chemicals. Preservative chemicals are selected such that they can preserve the characteristics of the urine analytes that are relevant to the analytical-principal. In certain non-limiting embodiments, if the analyzer being validated is image-based, and the reference analyzer is flow cytometry-based, then the preservative should preserve the morphological characteristics of the analytes (which are relevant to the image-based analyzer), as well as biochemical compositions of the analytes (which are relevant for the flow-cytometry analyzers). Non-limiting examples of such preservatives include formaldehyde-releasing chemicals (also referred to as aldehyde-releasing chemicals) such as imidazolidinyl urea or diazolidinyl urea. In certain embodiments, the preservative where the analyzer being validated is image-based, and the reference analyzer is flow cytometry-based, the preservative is not formaldehyde. In other non-limiting embodiments, if the analyzer being validated is an image-based analyzer, and the reference analyzer is also an image-based analyzer (or a manually performed imaged-based analysis), then the preservative should preserve the morphological characteristics of the analytes. Non-limiting examples of such preservatives may be selected from aldehydes such as formaldehyde or glutaraldehyde; or biomolecule fixatives such as alcohols, for example, ethanol.

Predetermined storage conditions may include a temperature at which the urine sample is stored prior to analysis. Urine sample (D) and urine sample (2) may be stored at a temperature of 0-10° C., or from 0-15° C., or from 0-20° C., or from 0-30° C., or from 5-10° C., or from 5-20° C., or from 5-30° C., or from 10-20° C., or from 10-30° C., or from 15-20° C., or from 0-30° C., or from 20-30° C. The predetermined storage conditions depend on the type of analyte that the new urine sediment analyzer is being validated for. To validate a urine sediment analyzer for accuracy and precision for cellular components (red blood cells, white blood cells, epithelial cells, or sperm), biochemical components (cast), and/or microorganisms (bacteria or yeast), urine sample (D) and/or urine sample (2) is stored at a temperature of 0-15° C. prior to analysis of urine sample (D) and/or urine sample (2). To validate a urine sediment analyzer for accuracy and precision for crystals, urine sample (D) and/or urine sample (2) is stored at a temperature of 15-30° C. prior to analysis of urine sample (D) and/or urine sample (2).

Validation of accuracy and precision determinations of a urine sediment analyzer may be performed during development of a new urine sediment analyzer, after instrument servicing/alteration, and/or at pre-defined intervals. The validity of accuracy and precision determinations of a urine sediment analyzer may be accomplished by the analysis of a urine sample by a reference urine sediment analyzer and the new urine sediment analyzer, and comparing the detection results obtained from the reference urine sediment analyzer and the new urine sediment analyzer. When the detection results of the new urine sediment analyzer fall within the acceptable values, a user can be assured that the new urine sediment analyzer is functioning properly and reporting accurately. However, when the detection results of the new urine sediment analyzer fall outside the acceptable values, a user should be alerted that the analytical results obtained from the new urine sediment analyzer may be inaccurate.

In one non-limiting embodiment of the presently disclosed and/or claimed inventive concept(s), a new urine sediment analyzer is validated by calculating the agreement between detection results of a urine sample analyzed by both the new urine sediment analyzer and a reference urine sediment analyzer. The urine sample is analyzed by both the new urine sediment analyzer and the reference urine sediment analyzer. Detection results from both analyzers are obtained and compared to determine any deviation between the two detection results. The accuracy and precision of the new urine sediment analyzer is validated if the deviation between the detection results does not exceed a predefined threshold level.

### NON-LIMITING EXAMPLES OF THE INVENTIVE CONCEPT(S)

A method for validating the accuracy characteristic determinations of an automated urine sediment analyzer, comprising the steps of analyzing one or more urine samples for one or more analytes of interest, using a first automated sediment analyzer within a time period of less than or equal to 10 hours from collection of the urine samples, wherein the urine sample is selected from (a) urine sample (A) is not subject to a temperature of below 8° C. prior to analyzing urine sample (A); (b) urine sample (B) has not had a preservative chemical applied to urine sample (B) prior to analyzing urine sample (B); (c) urine sample (C) was treated using predetermined separation techniques prior to analyzing urine sample (C); (d) urine sample (D) has had a preservative chemical applied to urine sample (D), and further wherein urine sample (D) was treated using predetermined storage conditions prior to analyzing urine sample (D); or (e) any combination of (a)-(d); obtaining detection results of the one or more analytes of interest for the one or more urine samples from the first automated sediment analyzer; analyzing the same one or more urine samples as analyzed by the first automated sediment analyzer for the one or more analytes of interest using a second automated sediment analyzer; obtaining detection results of the one or more analytes of interest for one or more urine samples from the second automated sediment analyzer; comparing the detection results of the one or more analytes of interest for the one or more urine samples from the first automated sediment analyzer and the second automated sediment analyzer to determine any deviation between the two detection results; validating the accuracy characteristic determinations of the first automated sediment analyzer by determining whether the deviation between the detection results of the first sediment analyzer and the second automated sediment analyzer exceeds a predefined threshold level.

The method, wherein the second sediment analyzer is an established reference analyzer.

The method, wherein the one or more analytes of interest can be selected from red blood cells, white blood cells, sperm cells, pathological casts, hyaline casts, bacteria, yeast, crystals, pus cells, squamous epithelial cells, non-squamous epithelial cells, transparent casts, and mucus.

The method, wherein the first sediment analyzer utilizes a detection technology selected from a flow cytometry-based method of detection or image-based method of detection.

The method, wherein the second sediment analyzer utilizes a detection technology selected from a flow cytometry-based method of detection, image-based method of detection, and a manual microscopy method of detection.

The method, wherein the second automated sediment analyzer utilizes a detection technology different than that of the first sediment analyzer.

The method, wherein the second automated sediment analyzer utilizes a detection technology that is the same as that of the first sediment analyzer.

The method, wherein the first sediment analyzer utilizes an image-based detection technology, and further wherein the preservative chemical is selected from biological cell preservatives such as aldehydes, aldehyde releasing chemicals, or biomolecule fixatives.

The method, wherein the first sediment analyzer utilizes a flow cytometry-based detection technology, and further wherein the preservative chemical is selected from biological cell preservatives such as aldehydes, aldehyde releasing chemicals, or biomolecule fixatives.

The method, wherein the one or more analytes of interest is selected from red blood cells, white blood cells, epithelial cells, sperm cells, pathological casts, hyaline casts, bacteria, or yeast, and further wherein the predetermined storage conditions include a temperature at which urine sample (D) is stored prior to analysis of urine sample (D), wherein the temperature is 0-10° C.

The method, wherein the one or more analytes of interest are crystals, and further wherein the predetermined storage conditions include a temperature at which urine sample (D) is stored prior to analysis of urine sample (D), wherein the temperature is 20-30° C.

The method, wherein the second sediment analyzer utilizes an image-based detection technology or a flow cytometry-based detection technology, and further wherein urine sample (C) is subjected to a separation technique prior to analysis of urine sample (C).

The method, wherein the second sediment analyzer utilizes a manual microscopy detection technology, and further wherein urine sample (C) is not subjected to a separation technique prior to analysis of urine sample (C).

A method for validating the precision characteristic determinations of an automated urine sediment analyzer, comprising the steps of analyzing one or more urine samples for one or more analytes of interest, using a first automated sediment analyzer two or more times within a time period of less than or equal to 10 hours from collection of the one or more urine samples, wherein the one or more urine samples are selected from (i) urine sample (1) has had a preservative chemical applied to urine sample (1); (ii) urine sample (2) was treated using predetermined storage conditions prior to analyzing urine sample (2); or (iii) any combination of (i)-(ii); obtaining detection results of the one or more analytes of interest for one or more urine samples from the first automated sediment analyzer for the two or more times that the first automated sediment analyzer was used to analyze the one or more urine samples; comparing the detection results of the one or more analytes of interest for the one or more urine samples from each of the two or more times that the first automated sediment analyzer was used to determine any deviation between the two or more times that the first automated sediment analyzer was used; validating the precision characteristic determinations of the first automated sediment analyzer by determining whether the deviation between the two or more times that the first automated sediment analyzer was used exceeds a predefined threshold level.

The method for validating the precision characteristic determinations of an automated urine sediment analyzer, wherein the one or more analytes of interest can be selected from red blood cells, white blood cells, sperm cells, pathological casts, hyaline casts, bacteria, yeast, crystals, pus cells, squamous epithelial cells, non-squamous epithelial cells, and mucus.

The method for validating the precision characteristic determinations of an automated urine sediment analyzer, wherein the first sediment analyzer utilizes a detection technology selected from a flow cytometry-based method of detection, image-based method of detection, and a manual microscopy method of detection.

The method for validating the precision characteristic determinations of an automated urine sediment analyzer, wherein the first sediment analyzer utilizes an image-based detection technology, and further wherein the preservative chemical is selected from biological cell preservatives such as aldehydes, aldehyde releasing chemicals, or biomolecule fixatives

The method for validating the precision characteristic determinations of an automated urine sediment analyzer, wherein the first sediment analyzer utilizes a flow cytometry-based detection technology, and further wherein the preservative chemical is selected from biological cell preservatives such as aldehydes, aldehyde releasing chemicals, or biomolecule fixatives.

The method for validating the precision characteristic determinations of an automated urine sediment analyzer, wherein the one or more analytes of interest is selected from red blood cells, white blood cells, epithelial cells, sperm cells, pathological casts, hyaline casts, bacteria, or yeast, and further wherein the predetermined storage conditions include a temperature at which urine sample (2) is stored prior to analysis of urine sample (2), wherein the temperature is 0-10° C.

The method, wherein the one or more analytes of interest are crystals, and further wherein the predetermined storage conditions include a temperature at which urine sample (2) is stored prior to analysis of urine sample (2), wherein the temperature is 20-30° C.

What is claimed is:

1. A method for validating the accuracy characteristic determinations of an automated urine sediment analyzer, comprising:
analyzing one or more urine samples for one or more analytes of interest, using a first automated sediment analyzer within a time period of less than or equal to 10 hours from collection of the one or more urine samples, wherein the one or more urine samples are selected from:
(a) urine sample (A) is not subject to a temperature of below 8° C. prior to analyzing urine sample (A);
(b) urine sample (B) has not had a preservative chemical applied to urine sample (B) prior to analyzing urine sample (B);
(c) urine sample (C) was treated using predetermined separation techniques prior to analyzing urine sample (C);
(d) urine sample (D) has had a preservative chemical applied to urine sample (D), and further wherein urine sample (D) was treated using predetermined storage conditions prior to analyzing urine sample (D); or
(e) any combination of (a)-(d);
obtaining detection results of the one or more analytes of interest for the one or more urine samples from the first automated sediment analyzer;
analyzing the same one or more urine samples as analyzed by the first automated sediment analyzer for the one or more analytes of interest using a second automated sediment analyzer;
obtaining detection results of the one or more analytes of interest for one or more urine samples from the second automated sediment analyzer;
comparing the detection results of the one or more analytes of interest for the one or more urine samples from the first automated sediment analyzer and the second automated sediment analyzer to determine any deviation between the two detection results;
validating the accuracy characteristic determinations of the first automated sediment analyzer by determining whether the deviation between the detection results of the first sediment analyzer and the second automated sediment analyzer exceeds a predefined threshold level.

2. The method of claim 1, wherein the second sediment analyzer is an established reference analyzer.

3. The method of claim 1, wherein the one or more analytes of interest can be selected from red blood cells, white blood cells, sperm cells, pathological casts, hyaline casts, bacteria, yeast, crystals, pus cells, squamous epithelial cells, non-squamous epithelial cells, and mucus.

4. The method of claim 1, wherein the first sediment analyzer utilizes a detection technology selected from a flow cytometry-based method of detection or image-based method of detection.

5. The method of claim 1, wherein the second sediment analyzer utilizes a detection technology selected from a flow cytometry-based method of detection, image-based method of detection, and a manual microscopy method of detection.

6. The method of claim 1, wherein the second automated sediment analyzer utilizes a detection technology different than that of the first sediment analyzer.

7. The method of claim 1, wherein the second automated sediment analyzer utilizes a detection technology that is the same as that of the first sediment analyzer

8. The method of claim 1, wherein the first sediment analyzer utilizes an image-based detection technology, and further wherein the preservative chemical is selected from biological cell preservatives such as aldehydes, aldehyde releasing chemicals, or biomolecule fixatives.

9. The method of claim 1, wherein the first sediment analyzer utilizes a flow cytometry-based detection technology, and further wherein the preservative chemical is selected from biological cell preservatives such as aldehydes, aldehyde releasing chemicals, or biomolecule fixatives.

10. The method of claim 1, wherein the one or more analytes of interest is selected from red blood cells, white blood cells, epithelial cells, sperm cells, pathological casts, hyaline casts, bacteria, or yeast, and further wherein the predetermined storage conditions include a temperature at which urine sample (D) is stored prior to analysis of urine sample (D), wherein the temperature is 0-10° C.

11. The method of claim 1, wherein the one or more analytes of interest are crystals, and further wherein the predetermined storage conditions include a temperature at which urine sample (D) is stored prior to analysis of urine sample (D), wherein the temperature is 20-30° C.

12. The method of claim 1, wherein the second sediment analyzer utilizes an image-based detection technology or a flow cytometry-based detection technology, and further wherein the urine sample (C) is subjected to a separation technique prior to analysis of urine sample (C).

13. The method of claim 1, wherein the second sediment analyzer utilizes a manual microscopy detection technology, and further wherein urine sample (C) is not subjected to a separation technique prior to analysis of urine sample (C).

14. A method for validating the precision characteristic determinations of an automated urine sediment analyzer, comprising:
analyzing one or more urine samples for one or more analytes of interest, using a first automated sediment analyzer two or more times within a time period of less than or equal to 10 hours from collection of the one or more urine samples, wherein the one or more urine samples are selected from:
(i) urine sample (1) has had a preservative chemical applied to urine sample (1);
(ii) urine sample (2) was treated using predetermined storage conditions prior to analyzing urine sample (2);
(iii) any combination of (i)-(ii);
obtaining detection results of the one or more analytes of interest for one or more urine samples from the first automated sediment analyzer for the two or more times that the first automated sediment analyzer was used to analyze the one or more urine samples;
comparing the detection results of the one or more analytes of interest for the one or more urine samples from each of the two or more times that the first automated sediment analyzer was used to determine any deviation between the two or more times that the first automated sediment analyzer was used;
validating the precision characteristic determinations of the first automated sediment analyzer by determining whether the deviation between the two or more times that the first automated sediment analyzer was used exceeds a predefined threshold level.

15. The method of claim 14, wherein the one or more analytes of interest can be selected from red blood cells, white blood cells, sperm cells, pathological casts, hyaline casts, bacteria, yeast, crystals, pus cells, squamous epithelial cells, non-squamous epithelial cells, and mucus.

16. The method of claim 14, wherein the first sediment analyzer utilizes a detection technology selected from a flow cytometry-based method of detection, image-based method of detection, and a manual microscopy method of detection.

17. The method of claim 16, wherein the first sediment analyzer utilizes an image-based detection technology, and further wherein the preservative chemical is selected from biological cell preservatives such as aldehydes, aldehyde releasing chemicals, or biomolecule fixatives.

18. The method of claim 16, wherein the first sediment analyzer utilizes a flow cytometry-based detection technology, and further wherein the preservative chemical is selected from biological cell preservatives such as aldehydes, aldehyde releasing chemicals, or biomolecule fixatives.

19. The method of claim 16, wherein the one or more analytes of interest is selected from red blood cells, white blood cells, epithelial cells, sperm cells, pathological casts, hyaline casts, bacteria, or yeast, and further wherein the predetermined storage conditions include a temperature at which urine sample (2) is stored prior to analysis of urine sample (2), wherein the temperature is 0-10° C.

20. The method of claim 16, wherein the one or more analytes of interest are crystals, and further wherein the predetermined storage conditions include a temperature at which urine sample (2) is stored prior to analysis of urine sample (2), wherein the temperature is 20-30° C.
